(19) United States
(12) Patent Application Publication
(10) Pub. No.: US 2012/0315639 A1
(43) Pub. Date: Dec. 13, 2012

(54) METHOD AND APPARATUS FOR SINGLE CELL ISOLATION AND ANALYSIS

(76) Inventors: GLENN YAGUANG DENG, San Jose, CA (US); Jie Zhang, San Jose, CA (US); Fang Tian, Palo Alto, CA (US)

(21) Appl. No.: 13/483,422

(22) Filed: May 30, 2012

Related U.S. Application Data
(60) Provisional application No. 61/494,478, filed on Jun. 8, 2011.

(51) Int. Cl. G01N 33/574; C12Q 1/68; C12M 1/42; G01N 33/566

(52) U.S. Cl. 435/6.12; 435/7.23; 435/7.21; 435/6.1; 435/309.1; 435/287.2

(57) ABSTRACT

A method and apparatus are disclosed here for rare target cell enrichment and isolation, where the captured target cells can be individually picked up and used for downstream analysis. This method and apparatus utilize antibodies conjugated microbeads to isolate target cells, use ON/OFF controls for the target cell capturing magnet and the release magnet, such that there is no need to change the cap of the capturing magnet and thus enabling automatic multiple rounds of capturing, washing and releasing cycles to increase the target cell detection sensitivity and reproducibility. A special filter is utilized to effectively remove more than 95% of free unbound microbeads, thus significantly improving the purity of the collected target cells and increasing the data quality of downstream analysis of the single target cells. Lastly, RNA expression patterns are proposed for identifying of certain target cells (e.g. circulating tumor cells and white blood cells).

### CROSS REFERENCE OF RELATED APPLICATIONS

The present application claims the benefit of U.S. Provisional Application No. 61/494,478, filed on June 8, 2011, which is hereby incorporated by reference in its entirety.

### STATEMENT REGARDING FEDERALLY SPONSORED RESEARCH OR DEVELOPMENT

None

### BACKGROUND OF THE INVENTIONS

1. Field of Invention

This invention relates generally to method and apparatus for cell isolation and analysis, and more specifically, method and apparatus for group cells isolation and single cell isolation which enables downstream analysis of the isolated group cells and single cells on an individual basis.

2. Background

Certain diseases tend to create specific type of cells (or target cells) which can be used as a biomarker in diagnosing and tracking the progression of a particular disease. For example, circulating tumor cells (CTCs) were reported more than a century ago by Thomas Ashworth [1]. However, there was not enough understanding about the properties of CTCs in both clinical and basic research because CTCs are rare cells and hard to isolate from their host samples for a while. Recently, it has been found that CTCs are present at a wide range of frequencies in patients with various metastatic carcinomas. As a result, the identification of CTCs helps physicians in monitoring and predicting cancer progression. It is also useful to the evaluation of a patient's response to therapy, especially for those patients with metastatic cancer. The number of CTCs in the blood has been shown to correspond to the clinical course of disease and is a predictor of patient's overall survival rate. Some clinical studies have shown correlation between CTC counts and progression of disease for certain types of cancer, such as metastatic breast cancer, colorectal cancer, and prostate cancer. Therefore, much efforts have been made in developing methods to capture and isolate biological cells. Better understanding about specific cells, such as CTC, are needed. Such understanding about CTCs could bring revolutionary treatment to cancer diseases. Therefore, cell isolation devices are helpful and sometimes even necessary to further advancement in the related areas, including, but are not limited to, life science research, healthcare study, and medical treatment. An isolated cell, after enrichment, isolation, and purification, can be used in subsequent downstream tests and measurements, such as analyzing DNA mutation and RNA/protein expression even at single cell level, understanding certain tumor formation mechanism and metastatic processes, detecting or monitoring various diseases, performing pathology analysis, documenting a person's identity, and classifying animal species.

One of the most commonly used methods is immunomagnetic cell enrichment using magnetic beads conjugated with specific antibody to specific cell and isolate target cells from various samples including blood, tumor tissues, biopsies, or bone marrow etc. Such methods rely on detection of CTCs by specific antibodies conjugated magnetic beads which are mixed with samples and then captured by a fixed magnetic rod covered with a plastic cap. Thereafter, the captured cells are usually cleaned by washing them in certain liquid solution to remove non specific cells and unwanted materials from samples. Then, the cells are released or separated from the plastic cap in order to be collected for cell counts or use it for further study.

CTCs are hard to detect using regular blood analysis methods and have been difficult to isolate until recently. Using CellSearch system (Johnson and Johnson), it was shown that CTCs are consistently present in the blood system of cancer patients. Scientists further demonstrated that CTCs have significant clinical values as prognostic markers using CellSearch system [2]. However, CTC detection still has not been adopted in routine clinical practice, such as American Society of Clinical Oncology (ASCO) guideline because greater sensitivity are needed to detect CTCs and more downstream analysis of CTCs are required to understand the properties of CTCs better.

In addition to CellSearch system, there are more magnetic enrichment technologies being developed, for example, techniques described in U.S. Pat. No. 3,970,518, Giaever et al, entitled “Magnetic Separation of Biological Particles”, U.S. Pat. No. 5,200,084, Liberti et al, entitled “Apparatus and Methods for Magnetic Separation”, U.S. Pat. No. 5,837,144, Bienhaus et all, entitled “Method of Magnetically Separating Liquid Components”, U.S. Pat. No. 8,071,395, Davis, entitled “Method and Apparatus for Magnetic Separation of Cells”, and techniques utilized by various products including AutoMACS separation (Miltenyi Biotec), Ariol (Microsystems), RoboSep (StemCell), and MagSweeper [3]. Using these technologies and products, EpCAM or Cytokeratin positive cells can be enriched and thereafter detected.

However, many problems still remain unsolved. One of the main issues is removing unbound free microbeads from captured target cells and this problem exists in essentially all presently known positive magnetic enrichment approaches. These unbound free microbeads obstruct target cell observations, single cell pick up, and DNA/RNS isolation. The free microbeads also reduce data quality in the subsequent down-stream analysis.

Another problem with known cell isolation methods and devices using magnetic probe is that in order to change the magnetic field strength at the tip of the probe for the purpose of alternatively capturing and releasing target cells enriched with magnetic beads, they require alternatively attaching and detaching the capture probe cap from the fixed magnetic probe. Such requirement of detaching the capture probe cap from the magnetic probe severely limits the effectiveness of target cell collection, reduces the sensitivity of cell isolation devices, and as a result hinders the adoption and development of advanced cell isolation techniques. Some approaches try to improve their sensitivity by repeating the capturing steps without releasing the captured target cells. However, capturing efficiency is only improved marginally by this approach, and it causes damages to target cells most time. Other approaches try to improve their cell capture sensitivity by changing the capture probe cap enclosing the magnetic probe before taking the next round of capture, wash, release cycle, but such procedures take too long, reduce the efficiency and device automation level.

Even if a rare cell has been isolated and collected, further analysis is required to determine whether the collected cells are indeed the target cells. Existing approaches are often too cumbersome to identify rare cells, such as CTCs.

Therefore, what is needed is a cell isolation method and apparatus that does not suffer from the aforementioned problems, a method and apparatus which efficiently removes unbound free microbeads from target cells, a method and apparatus that enables multiple round of cell capturing-release steps along efficient pre-determined patterns without the requirement of physically removing the capture probe cap from its magnetic probe, thus providing a high sensitivity method and apparatus in isolating target cell, even at an individual cell level, so that rare cells can be collected for down-stream analysis, as well as a convenient method to identify rare cells.

### BRIEF SUMMARY OF THE INVENTION

The present invention advantageously fills the aforementioned deficiencies by disclosing a method and apparatus in isolating target cells, even at a single cell level. This invention uses a magnetic probe, moving in certain pre-determined patterns driven by a chosen protocol, to automatically perform target cell capturing, washing, and releasing. The capture mechanism is based on the fact that certain antibodies recognize and bind to specific target cells, and the antibodies can be conjugated with various magnetic microbeads. These antibody-conjugated microbeads can incubate with the sample, such as blood or other biological materials, so that the target cells are bound by the microbeads-conjugated antibodies and form a complex comprising target cells, antibody, and microbeads. Such complex can be detected and captured by a magnet such as a magnetic probe. This invention also enables multiple rounds of capturing and releasing of target cells conveniently and cost effectively through an ON/OFF system of the capture magnetic field of the magnetic probe without the need to move the capture probe cap. In addition, the present invention enables the movement of the magnetic probe according to the apparatus system requirement and design, whether in circular motion, or in U-shape, comb shape, or other patterns, which pattern could be different for the capturing, washing or releasing step, in order to optimize the overall target cell isolation sensitivity and efficiency. Furthermore, the present invention carries an important functional protocol that effectively removes free unbound microbeads by placing a filter between the magnetic probe and target cells and allows repeated pick-up and release cycles in the sample well and release well, thus enables effective removals of non specific target cell and other materials (from blood, tissues, biopsies, or bone marrow) as well as unwanted magnetic particles, such as unbound free microbeads. After highly pure target cells have been collected, the present invention discloses a convenient method to identify and analyze whether the collected cells are target cells, such as CTCs. As a result, the present invention brings the benefit of a higher cell capture rate, more consistent cell collection results, and cleaner final collected single cells essentially free from free unbound microbeads, as well as a convenient method to analyze collected target cells.

The present invention relates to the method and apparatus of cell isolation with high sensitivity, capable of isolating and collecting a single cell from a biological sample. The apparatus comprises a magnetic probe, a robotic arm driving the probe automatically, and one or more plates holding combinations of sample well, rinse tank, and release well. The magnetic probe has a magnetic field at the probe tip to capture target cells, which magnetic force can be turned ON/OFF.

There are various mechanisms in switching ON/OFF of the capturing magnetic field and release magnetic field. In one method, the probe comprises a magnetic rod inside a thin capture probe cap, covering the magnetic rod when the rod is in its bottom resting position, acting as a barrier between the rod and the sample. The robotic arm holds the cap in a fixed position relative to the robotic arm throughout the entire capturing, washing, and releasing cycle. In one particular embodiment, the magnetic rod floats freely inside the cap but can move upwards to a distance where its magnetic field at the tip of cap is significantly reduced. A repulsive magnetic field from a strong exterior magnet, having an opposite magnetic polarity as that of the magnetic rod, is placed underneath the capture probe cap below the cell release well. Such repulsive magnetic field can be generated and turned on (“ON” state) or off (“OFF” state) by mechanically move an exterior magnet under the cell release well into or out of the vicinity of the cell release well. Alternatively, this can be accomplished by turning on or off electromagnetic current which produces a magnetic field with a reverse magnetic polarity as that of the magnetic rod. The repulsive magnetic field from the exterior magnet pushes the inner floating magnetic rod to move up to a position where the magnetic field at the tip of the capture probe cap generated by the inner floating magnetic rod is diminished significantly, while the magnetic field generated by the exterior magnet is much stronger. As a result, the strong magnetic force from the exterior magnet pulls the magnetic beads and target cells off the capture probe cap down to the bottom of the cell release well. The magnetic probe can then immediately be placed back into the sample tank to repeat the previous target cell capture process. The inner floating magnetic rod, due to its gravitational force, drops down automatically to sit in intimate contact with the bottom of the capture probe cap. The magnetic probe can be moved by the robotic arm along another predetermined search pattern and pick up any remaining magnetized target cells in the sample container along the way.

Instead of having a floating magnetic rod inside a capture probe cap, the magnetic rod could be connected with a control mechanism that can mechanically move the rod along the vertical directions. The capturing magnetic field at the tip of the cap can thus be turned on and off by moving the magnetic rod inside the cap down and up respectively, with the automatic control mechanism connected to the magnetic rod. Another approach is that the magnetic rod includes electromagnetic material. By switching on/off or increasing/decreasing the electrical current for the electromagnetic material, the corresponding magnetic field can be turned on or off. This is not an exhaustive list of past and current methods to turn ON/OFF the capture magnetic force. In addition, with the advancement of modern technologies, other methods in turning ON/OFF such magnetic field could emerge in the future.

As described above, the method and apparatus disclosed here is based on specific antibodies that can recognize and bind to the target cells. The antibodies are conjugated on the surface of microbeads. There are a few commercially available microbeads which can incubate with samples (blood or biological materials) so the target cells will be bound by antibody microbeads and form a complex consisting of target cells, antibody and microbeads. The complex can then be collected by the magnetic probe, which will also pick up unbound free microbeads along the way. The strong magnetic field at the tip of the cap attracts magnetic objects nearby. This magnetic field pulls the conjugated complex comprising target cells, antibodies, and microbeads, as well unbound free magnetic beads in its vicinity, toward the outer surface of the capture probe cap. As a result, target cells are captured onto the tip of the capture probe cap. The captured cells are then washed in a wash tank to remove unwanted contaminations, which process can be repeated multiple times as necessary. Thereafter, the target cell can be released from the cap by switching OFF the capture magnetic field at the tip of the capture probe cap.

During the cell capture process, the magnetic probe and the capture probe cap are controlled by an automatic robotic arm to move as one unit to move either vertically or horizontally or other predetermined search path (including but are not limited to moving in one direction or back and forth along an elongated rectangle slightly wider than the probe width, multiple rectangles, comb-shape, folk-shape, S-pattern, U-pattern, circular motion from inside to outside, circular movement from outside to inside, or combinations thereof) to gather target cells distributed in the sample without affecting the magnetic strength at the tip of the capture probe cap. After the magnetic probe completes its search path in the sample tank, it is placed into a wash tank to remove unwanted non-magnetic materials from the capture probe cap. The wash process can be repeated a few times as needed. Thereafter, the magnetic probe is placed into a target cell release well.

In one particular embodiment, the capture magnetic field is turned on and the robotic arm drives the magnetic probe in a sample tank, in this particular embodiment designed as a comb-shape with a width slightly wider than the width of the probe. The magnetic probe moves inward to the end of the first tooth of the comb and then moves outward to its starting point, thereafter moves in and out of the next adjacent tooth of the comb, and so forth, until all branches of the comb-shape sample tank has been fully covered. Along the way, the probe picks up magnetic materials in the sample tank, including the target cell complex and free unbound microbeads. Thereafter, the magnetic probe is placed into a wash tank, in this particular embodiment, designed as an S-shape stretched horizontally. The probe is put into the upper portion of the S-shape and moved left and right to rinse off unwanted sample material such as blood and bone marrow, then moved along the upper neck of the “S” shape into the middle section of the S-curve. The probe then moves left and right repetitively to rinse off remaining unwanted sample material. Since this portion of the wash liquid is somewhat isolated from those in the upper part of the S-curve, it is cleaner and thus more effective in washing off undesirable materials. Then the probe is moved along the lower neck of the S-curve to the bottom section of the S-curve. Then the probe is moved left and right repetitively. Similarly, this part of the liquid is cleaner than the previous two sections of the liquid and thus more effective in rinsing. Thereafter, the magnetic probe is put into a release well, where the capture magnetic field is switched into OFF position. As a result, the magnetic field at the tip of the capture probe cap is turned off. Meanwhile, the magnetic field generated by the exterior magnet is turned to ON position. As a result, the magnetic force from the exterior magnet pulls the magnet beads and target cells off the capture probe cap down to the bottom of the cell release well. Thereafter, if needed, the magnetic probe can then be immediately placed back into the sample tank with its capture magnetic field switched back to ON position, and repeat the previous target cell capture process. The magnetic probe can be moved by the robotic arm along the comb-shaped sample tank and pick up any remaining magnetized target cells in the sample tank along the way. The above process can be repeated as many times as required without removing the capture probe cap, thus making the target cell capture, wash, and target cell release cycle efficient, accurate, and economical.

In this particular embodiment, the present invention advantageously enables optimized search pattern for each step without ever needing to remove the capture probe cap from the magnetic rod, therefore, providing a process that yields faster results and higher sensitivity.

In another particular embodiment of the present invention, the magnetic probe is placed into a sample tank containing CTCs mixed with magnetic microbeads in the sample. By moving the probe back and forth along a 4 pronged folk shape sample tank with each pitch consisting of an elongated rectangle slightly wider than the probe width, the probe picks up magnetized CTCs as well as free unbound magnetic microbeads along its path and captures them onto the outside of the capture probe cap. The magnetic probe is then put into the wash tank shaped in an S-curve, moving back and forth in certain pre-determined patterns along the S-shape to rinse off non-magnetic impurities, and then placed into the first release well. Then the capture magnetic field for the magnetic probe is turned to “OFF” state, while the external magnet source is turned to its “ON” state. This will cause all the magnetized complexes of target cells, microbeads picked up by the capture probe cap to drop off the cap and fall into the first release well. Then, a filter with pores of a diameter smaller than the CTCs but larger than microbeads is placed between the released complex and the magnetic probe. Thereafter, the capture magnetic field for the magnetic probe is turned back to ON state while the external magnetic source under the first release well is turned to its “OFF” state. By moving the magnetic probe in certain patterns, including but are not limited to circular concentric motion, in certain pre-determined direction, either from outside edge toward center or from center toward outside edge, above the filter, unbound free microbeads are sucked up through the pores of the filter and stuck onto the magnetic probe. The target cells with bound microbeads cannot move up through the filter because they are bigger than the size of the filter pore. Then the magnetic probe is then placed into a waste well where the capture magnetic field is turned to OFF and the external magnet source is set at its “ON” state so that all the unbound free magnetic microbeads on the capture probe cap are pulled off the cap and deposited onto the bottom of the waste well. This process of pulling microbeads up through the pore of the filter onto the cap and then removing the microbeads into a waste well can be repeated several times until there are no or few microbeads being picked up by the magnetic probe. Then the filter in the first release well is removed, and the magnetic probe is put into the first release cell, where the capture magnetic field is turned back to ON state and the external magnetic source under the first release well is placed in its “OFF” state. The target cells will be picked up and attached onto the capture probe cap and 95% of the unbound free microbeads are found to have been removed. Then, as an option, the magnetic probe can be placed into a second wash tank in certain specific shapes, such as a U-shape, and moved back and forth to rinse off unwanted impurities further. The probe is then placed into the final cell release well. The capture magnetic field is turned to OFF state, and a strong exterior magnet placed underneath the capture probe cap below the final cell release well is turned to its ON state. As a result, the strong magnetic force from the exterior magnet pulls the target cells off the capture probe cap down to the bottom of the second release well. As needed, the magnetic probe can then be immediately placed back into the sample tank and repeat the above process again. This entire capture/wash/release process can be repeated multiple times without removing the capture probe cap from the magnetic rod to ensure that all the target cells in the sample have been gathered onto the capture probe cap and eventually are collected and released into the final cell release well.

In this particular embodiment, the present invention advantageously enables, among other things, picking up target cells during immunomagnetic incubation. In addition, there is no need to remove the capture probe cap during any of the capture, wash, and release cycle, and no need to remove the cap when such cycles need to be repeated many times, thus enabling the capture, wash, release process to be repeated as many times as needed in any order desired. If a target was not picked up in the first round of capture/wash/release cycle, it can be easily picked up in the next cycles without undue delay. There is no saturation binding on the magnetic cap because every time the captured targets on the cap will be released and then start new process of capturing. In addition, unbound free microbeads picked up by the magnetic probe are effectively removed in the first release well by the filter. As a result, the cell collection efficiency is significantly improved and thus provides an increased yield in cell collection with a much shorter time required to collect the cells from the sample with most microbeads removed from the final isolated cell.

In still another embodiment of the present invention, following similar process as described above, except that at the filter is placed into the first release well before the magnetic probe with the captured magnetic materials attached to the capture probe cap is placed into the first release well. Then the capture magnetic field is turned into the OFF state, while the external magnetic source under the first release well is turned to ON state. Since microbeads are smaller than the filter pores, they will pass through the filter and fall onto the bottom of the first release well. The target cells, on the other hand, will stay on top of the filter because they are bigger than the filter pores.

In still another embodiment of the present invention, following similar process as described above, except that the special filter is rolled into a small column shape and placed into the first release well. Then the magnetic probe with the capture magnetic materials attached to the cap is placed into the inside of the filter column, with the capture magnetic field tuned to OFF state, and external magnetic source under the first release well turned to ON state. Then all the magnetic material attached to the cap will fall onto the bottom of the first release well, inside the filter column. Then the external magnetic source is turned to OFF state and the magnetic probe is turned to ON state. A relative movement between the magnetic material deposited into the release well and the probe is caused through various mechanisms. One way is to have the robotic arm drive the magnetic probe in a circle around the outside of the filter column, and stay very close to the filter. Since free unbound microbeads are smaller than the filter pore, they'll pass through the filter pore and attach to the magnetic probe. On the other hand, since the target cells are bigger than the filter pore, they will stay inside the filter column and remain there throughout the process. Other methods include moving the filter and probe together in various patterns so that the sample stirs up the magnetic materials inside the column to enable free unbound microbeads be more easily attracted by the outside probe. Alternatively, both probe and filter can stay close to each other in stationary position but the release well rotates itself to cause relative movement between the magnetic materials inside the filter column and the probe so that free microbeads have more opportunity to escape through different pores of the filter onto the capture probe cap. As described above, the magnetic probe can dispose the free microbeads into a waste well and repeat such microbeads removal process multiple times until none or few microbeads are seen on the probe. Then, the capture magnetic field is turned on and the magnetic probe is placed in the inside of the filter column. Since the filter column has a small diameter, the probe can easily pick up the target cells, and move on to the next steps.

In these two particular embodiments, the present invention advantageously enables effective removal of free unbound microbeads from the captured complexes and provides a much cleaner, purer final target cell for easier and more accurate cell counting and down-stream analysis.

In still another embodiment of the present invention, following similar process as described above, except that at the beginning of the capture/wash/release cycle, a special filter is placed into the sample tank. The filter has pore sizes such that individual magnetic beads can easily pass through the pore opening but the target cells themselves are too big to pass through the pores of the filter. Then the magnetic probe is put into the sample tank with the capture magnetic field turned to ON state. The probe will move in a predetermined search pattern above the filter through-out the entire sample tank area. Since free unbound microbeads are smaller than the diameter of the pores of the filter, they will be pulled up through the pores of the filter, unto the surface of the cap. Then, the free microbeads attached unto the magnetic probe are removed into a waste well, similar to the process described above. This process can be repeated multiple times until none or very few free microbeads are being picked up by the magnetic probe. Then, processes described earlier for the entire cycle or multiple cycles of capture, wash, and release can be carried out.

In still another embodiment of the present invention, following similar process as described above, except that after sample has been incubated with antibody and magnetic microbeads initially in an alternative sample dish. Then a special filter is first placed into the actual sample tank at some distance above the bottom of the target cell wash tank. The filter has pore sizes such that individual magnetic microbeads can easily pass through the pore opening but the target cells themselves are too big to pass through the pores of the filter. Then, the sample is poured into sample tank. Thereafter, an exterior magnetic field below the sample tank is turned on. Since free unbound magnetic microbeads are smaller than the filter pore size, they are pulled through the filter pores, and fall onto the bottom of the sample tank. The target cells, on the other hand, will remain on the top of the filter because the target cells are too big to pass through the filter pore. As needed, the exterior magnetic field can be turned to OFF and ON a few times to cause redistribution of free microbeads so that more of them can pass through the filter pore. As an option, the robotic arm may drive the magnetic probe, with the capture magnetic field at OFF state, to move inside the sample, in order to stir up the sample and encourage more free unbound microbeads to pass through the filter pore and fall down to the bottom of the sample tank. This process can be repeated multiple times as needed. Thereafter, the capture/wash/release cycle can be carried out as described above.

In the above two particular embodiments, the present invention enables initial removal of free unbound microbeads and thus enables more effective capturing of the target cells during capture step with less interference from free unbound microbeads and advantageously provides an efficient method in isolating target cells with few free microbeads.

In still another embodiment, using QX Systems' CI-101 device, CTCs, DTCs, and WBCs (white blood cells) have been isolated in single cell form from tumor cell lines (T47D, SKBR3, MDA231, MCF7) and then isolated for single cell gene expression analysis. Human reference total RNA positive control (Ref) and no template control (NTC) were used. Single cells of T47D, SKBR 3, MDA 231, MCF7 cell line and single white blood cells, circulating tumor cells (CTC) and disseminated tumor cells (DTC) were used to conduct UBB (reference gene), CD45 (blood cell marker), CK19, and EpCAM gene expression analysis. We have tabulated results of the single cell RT-QPCR delta CT data (except human reference RNA). No expression is demonstrated by the result of 0 delta CT from negative control. The four type genes of UBB, CD45, CKs, and EpCAM were detected in Ref total RNA, but not in NTC. UBB expressed in all biological sample (except NTC); CD45 is clearly only expressed in Ref and white blood cell, not in single tumor cells. CD45's special gene expression property of only being found in gene expression for white blood cells and reference RNA samples, but not in CTCs can be used for tumor cell identification after target cells have been isolated from blood or bone marrow samples.

It is therefore an object of the present invention to provide a method and apparatus of cell separation which is capable of performing multiple capturing, washing, and releasing cycles automatically and efficiently using a magnetic probe with its magnetic field set at either ON or OFF state, without the need to remove or change the capture probe cap during the entire process, while an external magnetic source is set at either ON or OFF state to pull captured target cells off the capture probe cap during various steps of the capturing/washing/releasing cycle, thus yielding highly sensitive, accurate, faster and reliable cell isolation results.

It is another object of the present invention to provide a method and apparatus for cell separation which easily collects and removes essentially all of the free unbound magnetic microbeads from the final released target cell, by placing a special filter at various stages of the capturing/washing/releasing steps so that the final isolated target cells are pure, sensitive, reliable, and capable of collecting single cells which can then be used for subsequent analysis either for more accurate cell counting without inferences from free microbeads or more accurate other down-steam analysis.

It is also an objective of the present invention to provide a method and apparatus for cell separation with optimized target cell search, capture, washing, releasing, isolating, purifying steps by designing the probe to move along various optimal patterns and directions so that target cells can be collected and isolated in the most efficient and most sensitive way in the most pure form.

It is also an objective of the present invention to provide a convenient method to identify and analyze collected cells to determine whether they are indeed target cells by utilizing CD45's special gene expression property of only being found in gene expression for white blood cells and reference RNA samples, but not in CTCs.

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein:

FIG. 1 is a top view of one possible configuration of the cell isolation device. In this particular configuration, the cell isolation device has two magnetic probes driven by the robotic arm, three standard six-well culture plates, six capture probe cap holders, and a waste box to remove and collect used caps. An automation system controlling the magnetic probe movement and setting the capturing magnetic field at ON/OFF state and an external magnetic source being at either ON or OFF state are not shown here.

FIG. 2 is a top view of a new design of magnetic probe movement pattern at various steps of the capturing, washing, and releasing cycle. In this particular design, the cell isolation device can process two samples at the same time. The capturing search tank is a four-toothed comb shape, the washing tank an S-shape, a second washing tank a U-shape, and two release wells in circular shape, sizes are not to scale.

FIG. 3 is a side view of the cell isolation device described in FIG. 1 with the capturing magnet turned to ON state and external magnet switched to OFF state, and with the capturing magnet turned to OFF state and external magnet switched to ON state, respectively.

FIG. 4 is a view in perspective of a magnetic probe inside a sample tank and one of its search patterns. The magnetic probe moves to the edge of the sample tank first, then moves in a concentric circle motion either clock-wise or counter-clock-wise toward the center. This is more effectively that searching by moving from center toward the edge in a concentric circle.

FIG. 5 are pictures of the actual magnetic probe and special filter used for cell isolation, with a) being the magnetic rod inside the magnetic probe; b) capture probe cap; c) magnetic rod with the capture probe cap on, forming the magnetic probe; and d) top view and side view of the special filter and filter holder.

FIG. 6 is a view in perspective of the cell isolation device with a magnetic probe in the entire capturing, washing, and releasing process, and repeated when necessary, without any need to change or remove the capture probe cap.

FIG. 7 is view in perspective of the cell isolation device with its capturing magnetic field turned to ON/OFF state.

FIG. 8 is a view in perspective explaining the mechanism of the special filter where the microbeads are smaller than the filter pore size while the target cell is bigger than the size of the filter pore.

FIG. 9 is a view in perspective of the cell isolation device shown in FIG. 7 including a special filter with core size of 8 um.

FIG. 10 is a figure of target cell collected into the release well with more than 95% of the free unbound microbeads removed effectively by the special filter. 10A is a picture of the magnetic probe captured material released into the final release well. 10B is the microscopic view of the captured target cells with most free microbeads still present blocking the view of actual target cell. 10C is microscopic view of the captured target cells with more than 95% of free microbeads removed after using a filter and clearly revealing the target cell.

FIG. 11 shows an example of cell identification using immunostain method with specific biomarkers. 11A: cytokeratins positive (FITC) cells; 11B: cell nuclear staining by DAPI; 11C: cells positive for CD45 (blood cell specific marker); 11D: merged image of cytokeratins, DAPI and CD45.

FIG. 12 shows the summary of a single cell expression analysis.

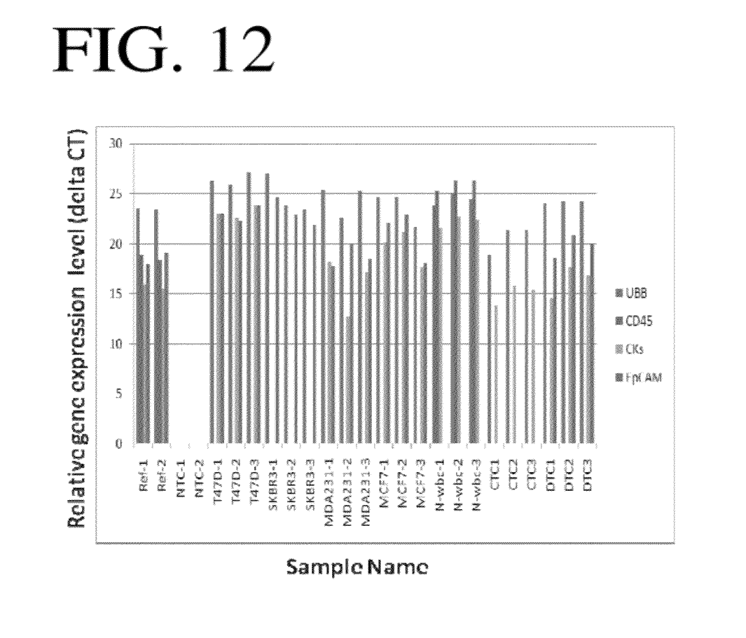

TAB. 1 summarizes a single cell RT-QPCR delta CT data for FIG. 12.

### DETAILED DESCRIPTION OF THE DRAWINGS

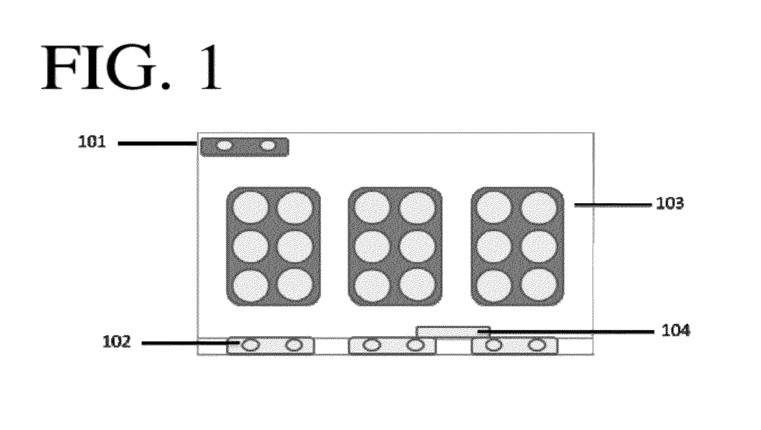

Referring now to the drawings wherein the showings are for purposes of illustrating a preferred embodiment of the invention only and not for purposes of limiting the same, FIG. 1 shows some of components for the cell isolation device, a magnetic probe holder 101 with two magnetic probes, six cap holders 102, a standard six-well culture plate 103, and a waste box 104 for discarding used caps from the magnetic probes.

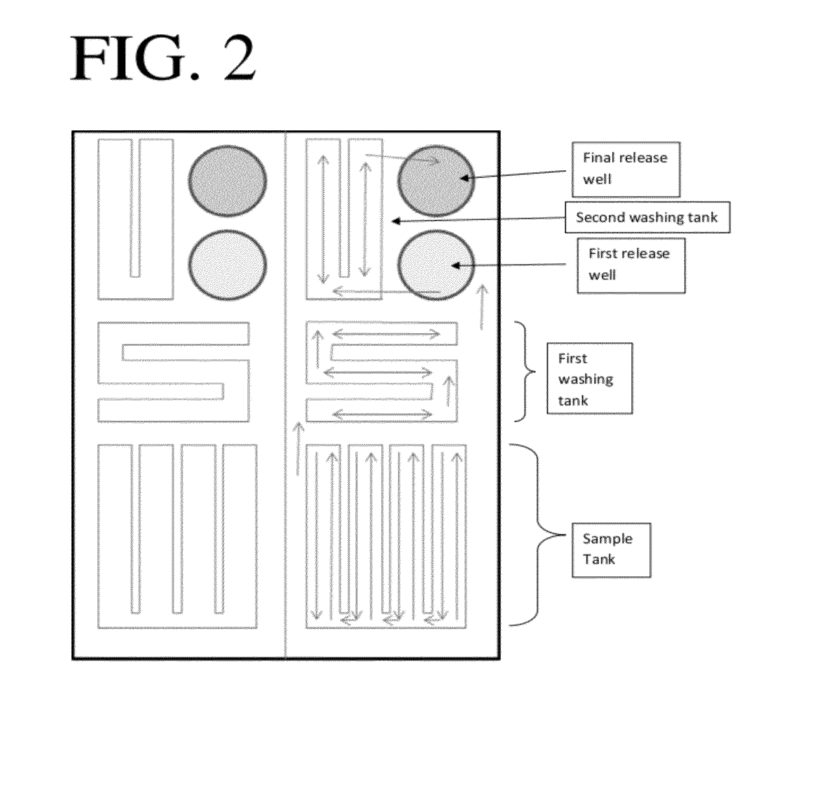

The scanning pattern for the magnetic probe can vary for different steps of the capturing, washing, and releasing cycle, in order to optimize the sensitivity, efficiency, reproducibility, and purity of the final collected target cell. FIG. 2 shows a new maze plate designed for CI-101 cell isolator. It can process two samples at the same time which fits well with the CI-101 two-magnetic probe structure. After a sample is incubated with antibody conjugated microbeads, the mixture is poured into the sample tank. Then the probe will scan the sample in the sample tank according to programmed routs (shaped as a comb). Then it will move to the first wash tank (shaped as an “S”) and washes the captured material in the buffer, and then releases the captured materials into the first release well filled with fresh buffer. After one round or multiple rounds of capturing/washing/releasing cycle, the used cap covering the magnetic rod will be removed and discarded. A new cap will be put on the magnetic rod. The magnetic probe will then pick up the released material from the first release well and perform an additional washing (shaped as an “U”) and finally release the captured targets into the final release well which is filled with fresh buffer.

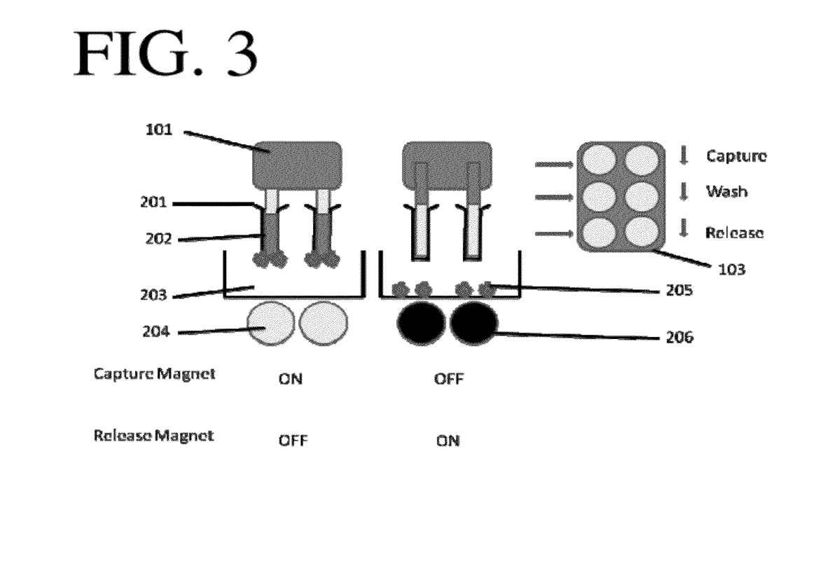

FIG. 3 illustrates the ON/OFF states for the capturing magnetic field and ON/OFF states for the external magnetic field. The magnetic probe holder 101 holds two magnetic rods 202 covered by the capture probe cap 201. In the capturing position 203, the capturing magnetic field is at its ON position 204 and the external magnet field (or release magnet) is at its OFF position 204. Therefore, magnetic materials in the sample, such as microbeads and target complex 205 will be picked up and bound to the surface of the cap 201. When the capturing magnetic field is turned OFF shown as 206, and the external magnetic field is turned ON also shown as 206, the microbeads and target complex bound to the surface of the cap will be released into the well. Magnetic probes will first scan the target cells in the sample well to capture them, then move to the washing well to wash off impurities and then release the collected materials into the release well. The capturing well, wash well, and release well positions are shown, as one of the possible configurations, as 103.

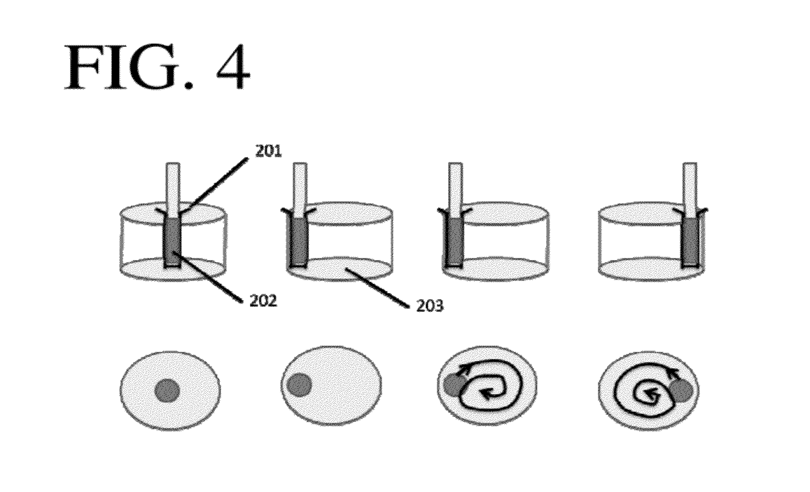

FIG. 4 illustrates a capturing step scanning routes where the magnetic probe moves in a concentric circular pattern, either clock-wise or counter-clock-wise. The magnetic probe first moves to the sample well for capturing, then move to the edge of sample well and starts scanning for target cell from outside gradually moving in concentric circular pattern toward the center of the well. For multiple capturing process, the probes can alternative between clock-wise and counter-clock-wise to optimize target cell collection efficiency and sensitivity.

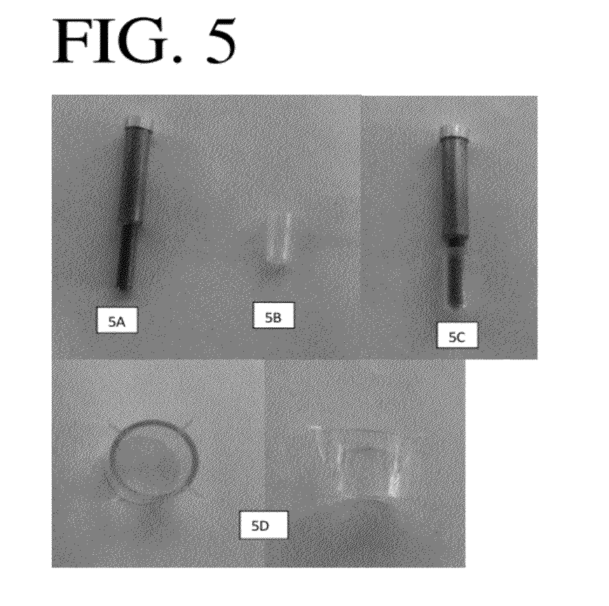

FIG. 5 shows a specially designed magnetic probe and capture probe cap for target cell capturing, washing and releasing, as well as a special filter for purification of captured material. During operation, the capture probe cap shown in 5B is put on the magnetic probe 5A, as shown in 5C. After collected materials are released into the first release well, filter 5D is put on top of the collected materials, close to but not touching the collected materials. The magnetic probe will then perform a capturing procedure to collect free unbound microbeads and remove them from the first release well. This removes more than 95% of the free unbound microbeads and provides a much purer isolated target cells.

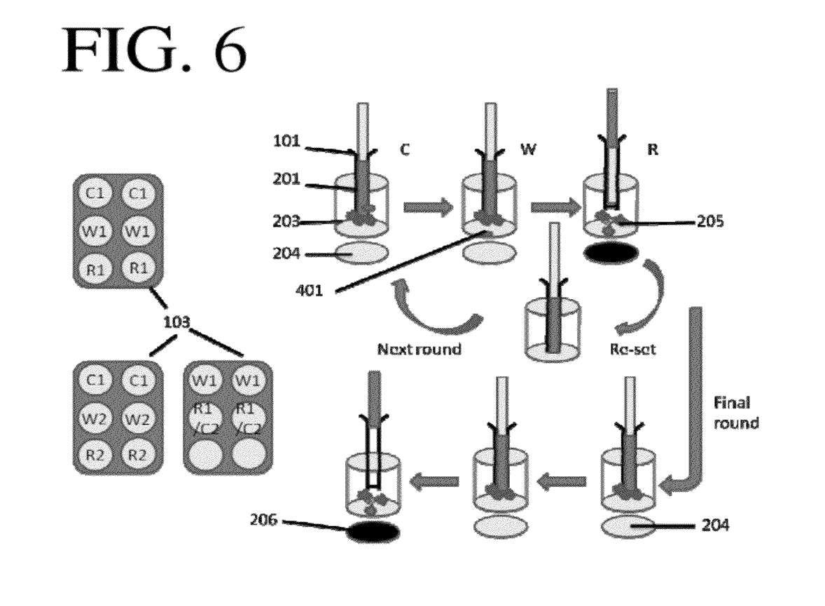

FIG. 6 illustrates how a cell isolation device conducts multiple rounds of capturing, washing and releasing cycle without the need to remove the capture probe cap unless there is a need to remove non-magnetic material bound to the surface of the cap. Cl is the first capturing, W1 the first washing, R1 the first releasing, C2 the second capturing, W2 the second washing, and R2 the second release. Magnetic probe 101 with cap 201 on, in its capturing position 203, and with capturing field in ON position 204, performs the first capturing, C1. The probe collects magnetic materials onto the surface of 201, also collects some non-magnetic impurities such as blood, bone marrow, along the way, shown as 401. The probe then moves to first washing position W1, washes off impurity 401, then moves on to first release position R1. The external magnetic force for R1 is then turned to ON state 206, magnetic materials 205 collected by the probe will drop into the first release well R1. At this stage (or at the end of multiple rounds of the capturing/washing/release cycle), if there is a need to remove non-magnetic material bound to the surface of the cap 201, the probe can be programmed to discard the old cap 201 and automatically put on a new cap 201. Then, probe 101 is reset to its capturing field ON position 204 and moves back to the first release well where external magnetic force is turned to OFF state 204, and performs a second capturing C2 to pick up the magnetic materials released during the first release R1. The probe 101 then moves to the second wash well, W2, and then proceeds to the second release well R2 to release the collected magnetic material.

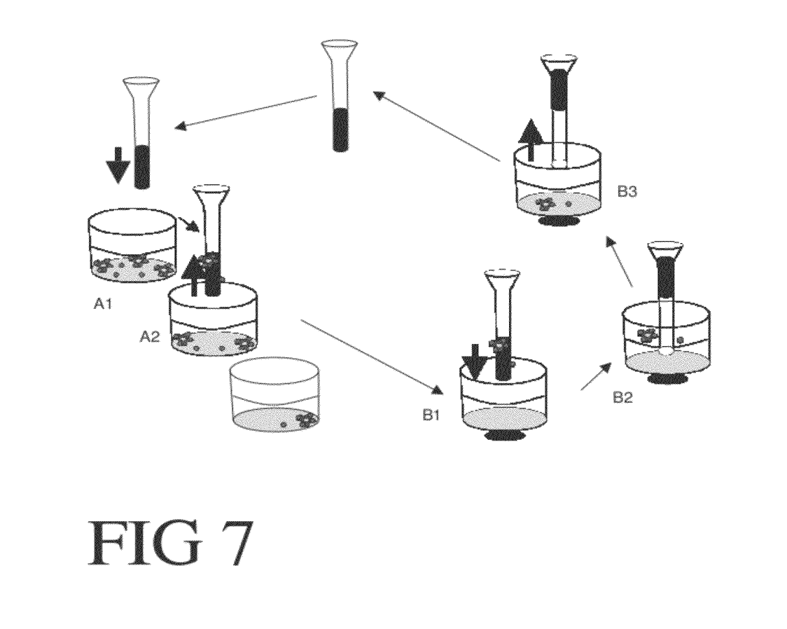

FIG. 7 is another perspective of the above process performing multiple rounds of the capturing, washing, and releasing cycle having the capturing magnetic field at ON/OFF states. A is the sample well, while B is releasing well. When the probe is put into the sample well initially, shown as A1, the probe picks up magnetic materials in the sample. The probe then moves out of sample well, perhaps with some target materials remaining in the sample well, shown as A2, then the probe moves into release well B1, which has the external magnet below the well turned on which will turn off the probe capturing magnetic field to OFF, then enabling the probe to drop collected materials into release well, shown as B2. Then, the probe is free from magnetic materials on its surface, shows as B3. Then, the probe can move back into the sample well and collect any remaining target cells and drop them into the release well.

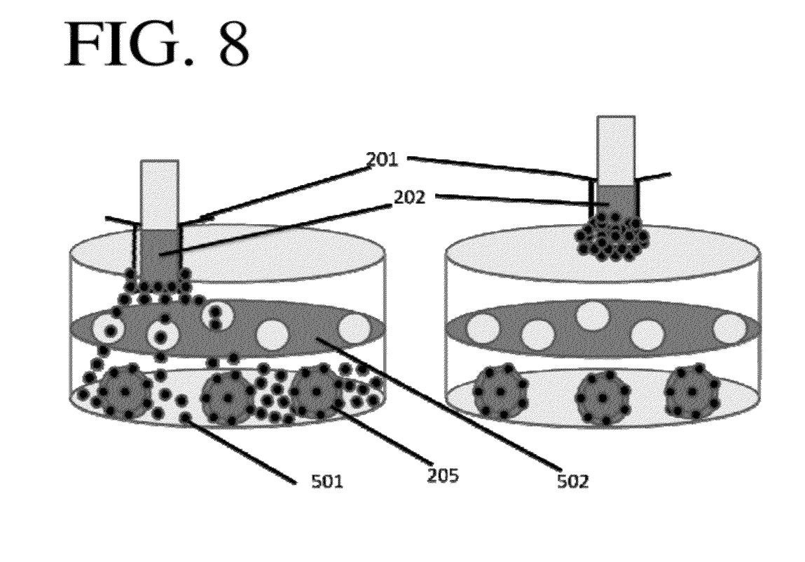

FIG. 8 demonstrates the mechanism by which the special filter effectively removes free unbound microbeads. A special filter, 502, with a core size bigger than the microbeads 501, and smaller than target cell complexes 205, is put over the captured materials released into the release well. In this particular embodiment, the filter core size is 8 um and microbeads size is 4.5 um, and target cells are greater than 10 um. The magnetic force from magnetic rod 202 will pull microbeads 501 through the pores of special filter 502, and pull them onto the surface of cap 202. Since target cell complexes 205 are bigger than the cores of the filter 502, they will remain intact under the filter 502.

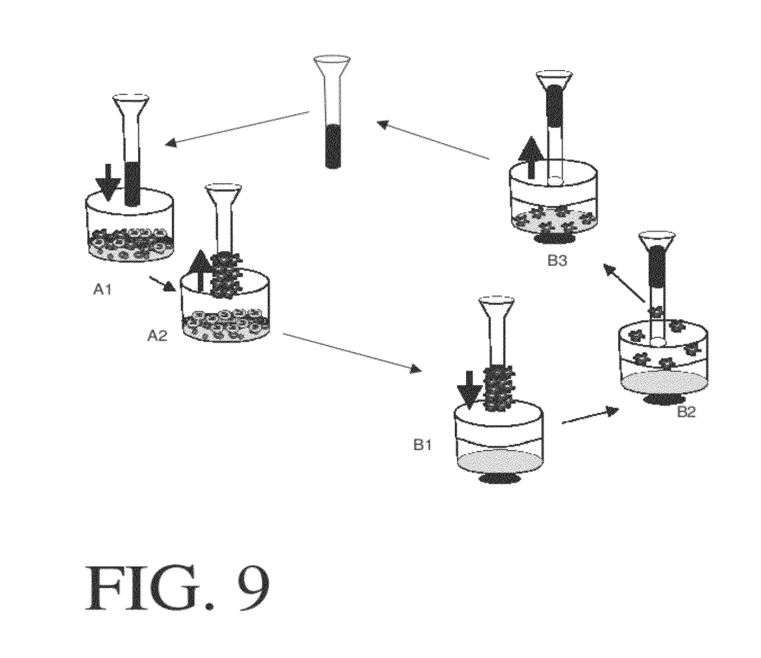

FIG. 9 is another perspective of the process illustrated in FIG. 7 except that a filter with a core size of 8 um is placed into the blood sample in the sample well A1. Microbeads and other cells smaller than the core of the filter will be partially filtered out because they will drop to the bottom portion of the sample well below the filter, while target cells will stay on top of the filer because they are bigger in size.

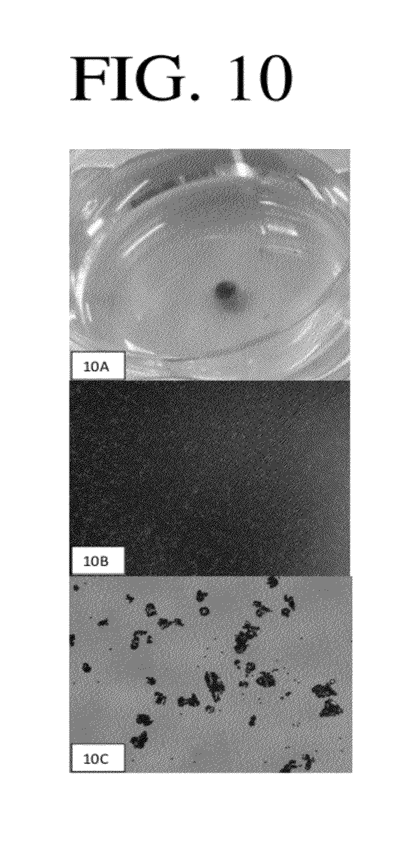

FIG. 10 shows the regular picture (FIG. 10A) and microscopic pictures of collected target materials without (FIG. 10B) and with the filter (FIG. 10C). Typically a large amount of free unbound microbeads are present in the final collected material, making it difficult to count target cells or perform downstream analysis of the target cells. More than 95% of free unbound microbeads are removed from the final collected material by using a filter. Under the microscope, target cells are not visible in 10B due to the large number of free microbeads. After removing most microbeads, target cells became clearly visible under the microscope as shown in 10C.

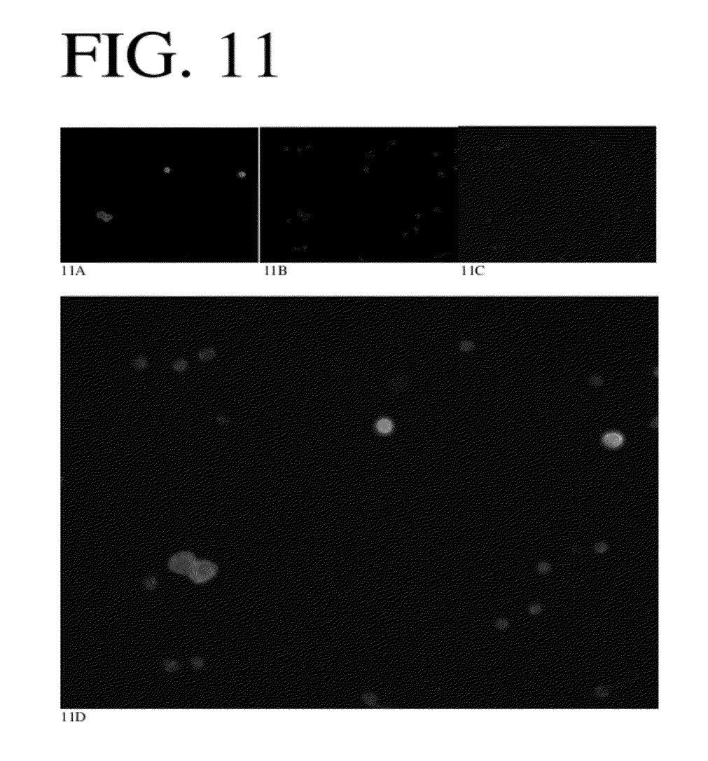

FIG. 11 shows an example of cell identification using immunostain assay with specific biomarkers. 11A: tumor cells show positive for cytokeratin (FITC); 11B: cells show positive for nuclear staining (DAPI); 11C: blood cells show positive for CD45 (TexasRed); 11D: a merged images from cytokeratins, CD45 and DAPI staining, indicating that blood cells display positive signals for CD45 and DAPI, tumor cells display negative signals for CD45, but positive for cytokeratins and DAPI.

FIG. 12 is a single circulating tumor cell expression analysis data. Human reference total RNA positive control (Ref) and no template control (NTC) were used. Single cells of T47D, SKBR 3, MDA 231, MCF7 cell line and single white blood cells, circulating tumor cells (CTC) and disseminated tumor cells (DTC) were used to conduct UBB (reference gene), CD45 (blood cell marker), CK19, and EpCAM gene expression analysis. CD45 was detected only in human reference total RNA and white blood cells.

TABLE. 1 is tabulated results of the single cell RT-QPCR delta CT data (except human reference RNA) for FIG. 12. No expression is demonstrated by the result of 0 delta CT from negative control. The four type genes of UBB, CD45, CKs, and EpCAM were detected in Ref total RNA, but not in NTC. UBB expressed in all biological sample (except NTC); CD 45 is clearly only expressed in Ref and white blood cell, not in single tumor cells.

### DETAILED DESCRIPTION OF THE INVENTION

The present invention is directed to the method and apparatus of isolating a cell, even at a single cell level, using a magnetic probe. The device described here are for enrichment, isolation, and purification of target cells such as circulating tumor cells (CTCs) or disseminated tumor cells (DTCs). The device can be used for rare cell isolation and purification for various types of samples and complete the entire cell capturing, washing, and releasing cycle, or multiple rounds of the cycle, efficiently because there is no need to change or remove the capture probe cap through-out the entire process. Such simplified process enables additional flexibility, and improves sensitivity and reproducibility for rare cell isolation and purification. The advantage of such device in providing high purity target samples do not just apply to rare target cell, they can be used in other applications as well, such as pharmaceutical and food industry, or other industries where small quantity of material needs to be detected or analyzed.

The following describes in detail the steps used for one of the cell isolation device configurations and associated procedures for each step. The magnetic probe is first moved to the cap holder to put the capture probe cap onto the magnetic rod, thereafter the probe is moved back to its home position so that its height can be calibrated. Then the probe is moved into the sample tank to perform target cell capturing scanning, then moved to washing tank to wash off impurities, then moved to releasing well for target cell collection. Thereafter, downstream analysis can be performed.

The cell capturing is a process of positive magnetic isolation of target cells which have been identified by certain antibodies conjugated with magnetic microbeads. The antibody conjugated microbeads are incubated with a sample which contains target cells. These microbeads can bind target cells through antibody specific binding and form a complex comprising target cells, microbeads, and antibodies. When the magnetic probe is moved close to such a complex, the magnetic force from the magnetic rod will attract the complex to the surface of the capture probe cap. When the magnetic probe is put into the washing tank, the magnetic force from the magnetic rod will continuously hold the complex and other magnetic materials onto the tip of the capture probe cap, while other impurities such as blood and bone marrows will be washed away while the probe moves through the washing buffer (e.g. PBS buffer). Then, the magnetic probe is placed into the release well. A strong external magnet is placed under the release well. It is designed such that at the tip of the capture probe cap the magnetic force from the external magnet is in opposite polarity to that from the magnetic probe. When the magnetic probe is placed above the external magnet, the strong magnetic force from the external magnet will push the magnetic rod up, effectively switching the magnetic probe into the OFF state and magnetic materials attached to the surface of the cap will be pulled off the cap and drop into the release well and fall into the release buffer (e.g. PBS buffer). This cycle of capturing, washing, and releasing can be repeated multiple time, in various combination, and eventually the target cell is released into the final release well. The robotic arm will remove the used capture probe cap and discard into a waste box holder. Thereafter, a new cap is put onto the magnetic rod and more capturing, washing, and releasing cycles can begin.

In another configuration, the robotic arm holds two magnetic probes. In this case, one cycle of capturing, washing, and releasing can be carried out in one six-well plate. However, if multiple rounds of capturing, washing, and releasing cycles are needed, two six-well plates are required to carry out all the steps. QX System's cell isolator device, CI-101 system, is capable of supporting three six-well plates configuration so the entire procedure can be completed automatically without any manual re-set up.

For a device configuration with a single six-well plate, one round of capturing, washing, and releasing cycle can be carried out easily and conveniently. This configuration can be particularly useful when relatively abundant target cells are present in the sample. Using the method and apparatus disclosed by this invention, effective capturing and isolation of a specific type of target cells can be carried out first, then capturing and isolation for another specific type of target cells can be carried out next until different types of target cells are all captured and isolated. This effectively provides a cell sorting function as well as cell counting. Furthermore, down-stream cell analysis can be performed on each type of target cells.

For rare targets, greater sensitivity and reproducibility are achieved by adding a few additional functionalities to the cell isolation device. One of the approaches the present invention discloses is adjusting the maximum magnetic force at the tip of the capture probe cap, generated by the magnetic probe, based on enrichment needs and this may vary at different steps of the capturing, washing, and releasing cycle. For example, if the sizes of microbeads are relatively large, a weaker magnetic force is chosen for the capturing process. If the sizes of the microbeads are small, a stronger magnetic force is chosen but not to the level at which the magnetic force damages target cells. Another approach is to optimize the scanning route. If the magnetic probe scans a sample in a circular motion, the present invention starts the probe from the edge of the well and then moves the probe either clock-wise or counter-clock-wise toward the center of the well. This rout is found to be more advantageous and provides better sensitivity and reproducibility compared to starting the probe at the center of the well and then moving the probe in circular motion toward the edge of the well. The sensitivity and reproducibility is further improved with our specially designed search pattern for each step of the capturing, washing, and releasing cycle. In addition, further improvement in sensitivity is obtained utilizing our multiple rounds of capturing/washing/releasing cycle. For a blood sample, one cycle is typically not sufficient in capturing target cells, such as CTCs, efficiently. We found that multiple cycles, typically three rounds of capturing/washing/releasing works well. Traditional methods use the probe to scan a sample once and then repeat the same scanning of the sample without changing the scanning route or replacing the capture probe cap. The problem with the traditional methods is that target cells with fewer microbeads are often not picked up in the first cycle and will have even less a chance to be picked up in the second scan if the magnetic probe is already covered with target cells, microbeads or other magnetic materials. The present invention solves such problems because the magnetic rod inside the magnetic probe can move up and down into its OFF/ON state easily, thus releasing target cells from or attracting target cells to the probe without the need to change the cap. With such a feature, after the first round of capturing/washing/releasing, the surface of the cap can be emptied out of magnetic materials without changing the cap by turning OFF the capturing magnetic field, and thereafter the cap can be used effectively for the next round of picking up target cells. The present invention also allows automatic cap changing in the middle of the capturing/washing/releasing cycle. For example, after the magnetic probe releases magnetic materials into the first release well, the old cap be discarded into the waste box and a new cap can be put onto the magnetic probe. This helps to avoid or reduce contamination from non-magnetic materials among the final isolated target cells. With a new cap, the probe can then continue to proceed to next steps of the process.

The present invention is highly effective in removing unbound free microbeads from captured magnetic material while keeping the target cell intact. Positive magnetic enrichment for rare target cells typically requires a large amount of microbeads in order to obtain high cell capture sensitivity and reproducibility. Therefore, after a standard capturing, washing, and releasing cycle, a large amount of unbound free microbeads are present in the final captured material, with the microbeads volume often significantly exceeding that of the target cells, thus making it difficult to either count target cells or perform further down-stream analysis of the target cells. One of the down-stream analyses is studying the DNA or RNA of isolated target cells. When large amount of microbeads are present with a rare target cell, the DNA and/or RNA isolation efficiency will be low and DNA/RNA quality will be poor. With the present invention, the magnetic probe captures target cells and free unbound microbeads, washes off impurities, then releases the collected materials into the first release well. Then, a filter with a pore size that is bigger than microbeads (e.g. 8 um of filter pore from Millipore), but smaller than the target cells is placed over the collected materials but not touching the materials (keep the distance at 1-2 mm). The magnetic probe then scans along a pre-determined pattern above the filter and picks up free unbound microbeads, removes them into a waste well, and repeats this process as many times as needed. More than 95% of the free unbound microbeads are removed during this process and the target cells are kept intact. This process enables collection of highly pure target cells and is particularly critical to isolating rare target cells (such as CTCs).

Using QX Systems' CI-101 device, single cells of CTCs, DTCs, and WBCs isolated from patient samples and single cells isolated from tumor cell lines (T47D, SKBR3, MDA231, MCF7) were used for single cell gene expression analysis. CD45 was only found in gene expression for WBCs and reference RNA samples, but not in tumor cells, while cytokeratins, EpCAM were expressed in tumor cells. This gene expression property can be used for tumor cell identification after target cells have been isolated from blood or bone marrow samples.

### General Procedures and Robot Control

Including the robotic control, the device working procedures include a machine initialization step to check, using photo comparisons, whether the sample plates are in place, the capture probe cap is on the magnetic probe, and the device door is closed. FIG. 1 shows some of the device components including the capture probe holder holding two magnetic probes, the cap holders, sample plates, and waste box. The magnetic probe moves to the cap holder and the robotic arm puts the cap onto the magnetic probe. A software program then adjusts the height of the magnetic probe and then performs the capturing, washing, and releasing process, either for a single cycle, or multiple cycles according to the protocol selection. Free unbound microbeads are removed from the target cell release well by placing a special filter over the target cells (not touching the target cells). Target cells in the final release well are then collected for observation under a microscope and then prepared for downstream analysis.

### CI-101 Parameters

Different strength of magnetic force from the magnetic rod can be chosen for specific target cell isolation. An exemplary configuration includes a magnetic rod made from a permanent magnet with a diameter of 0.5 cm and a length of 2.5 cm. The probe scanning speed can be adjusted as need, usually at 1-5 mm per second for target cell capturing step, and 2.5-30 mm per second for washing step, while keeping the probe distance from the bottom of the sample tank or wash tank between 0.5 mm to 1 mm. The path of each scanning route is kept at about 1-3 mm separation from previous path so there are enough overlap to not miss potential target cells.

### Single Cell Analysis

With CI-101, captured target cells can be purified by removing free unbound microbeads by filter purification. Then single cells can be picked up for downstream analysis using DNA and RNA markers. For example, the biomarkers of EpCAM, CK8/18/10 and CD45 can be analyzed for CTC identification using RT-QPCR. A target cell with CD45 positive signals is not considered a CTC. However, a target cell from cancer patients' blood sample with positive EpCAM and CK8/18/19 signals, but negative for CD45 will be considered as CTCs for tumor cell analysis.

### EXAMPLES

Examples below are for the purpose of providing references in demonstrating the principle of this invention only. The subject matter is not limited to these examples.

### Example 1

### Target Cell Isolation with One Round of Capturing, Washing, and Releasing Cycle and Completed in a Standard Single Six-Well Cell Culture Plate

MCF7 cells were cultured with DMEM media (Invitrogen) and harvested with Triple (LifeTech). Put approximately 1000 harvested MCF7 cells into approximately 10 mL of DMEM media in a 15 mL centrifuge tube, added 10 uL of EpCAM microbeads (Dynal), and then incubated between 30 minutes to 1 hour with a rotator at 4° C. (can be at other required temperature as desired). Poured the mixed sample into sample well, filled the other two wells (wash well, release well) with 10 mL of PBS buffer (pH=7.2) (can be any other buffers that fit the Protocol). The machine (QX Systems' CI-101) proceeded to perform the capturing, washing, and releasing cycle and finished in less than 10 minutes and provided relatively pure EpCAM positive MCF7 cells together with microbeads when released into the PBS buffer of the release well. These cells were picked up individually for single cell DAN or RNA analyses. (FIG. 12, TAB. 1).

### Example 2

### Target Cell Isolation with Multiple Rounds of Capturing, Washing, and Releasing Cycle

Put approximately 1000 MCF7 cells into a 10 mL normal human blood, added 10 uL of EpCAM microbeads (Dynal), and incubated for 1 hour with a rotator at 4° C. (can be at other required temperature as desired). Poured the mixed blood sample into the first well at one end of the standard six-well plate, filled other wells with PBS buffer (can be any other buffers that fit the Protocol). Added a second standard six-well plate and fill all with PBS buffer. Set up QX Systems' CI-101 to perform multiple rounds of capturing, washing, and releasing cycle for MCF7 isolation from the blood sample. The CI-101 machine automatically performed three times of capturing, washing, and releasing cycles and then the captured cells are released into the first release well. After the machine finished two rounds of capturing/washing/releasing cycle, to avoid contamination by non-magnetic materials stuck onto the capture probe cap, CI-101 automatically removes the used cap and puts a new cap onto the magnetic probe before the final round of the capturing/washing/releasing cycle. These target cells can then be observed under a microscope and can also be picked up individually for single cell downstream analyses. (FIGS. 6, 10). The well position for capturing, washing, and releasing can be changed to different positions as desired.

### Example 3

### Target Cell Purification by Removing Free Unbound Microbeads from Collected Materials

A 10 mL blood sample from a breast cancer patient was collected and put into an EDTA blood collection tube at room temperature. 10 uL of EpCAM microbeads (Dynal) was added to the blood sample and incubate at room temperature for 1 hour with mild rotation. The blood sample was then poured into the first well at one end of the first standard six-well plate. The other wells in the first standard six-well plate and the second standard six-well plate were filled with 10 mL PBS buffer solution. Set up QX Systems' CI-101 to perform super clean isolation Protocol. The CI-101 machine will automatically perform three rounds of capturing, washing, and releasing cycles and then the captured cells are released into the first release well. After the machine finishes two rounds of capturing/washing/releasing cycle, to avoid contamination by non-magnetic materials stuck onto the capture probe cap, CI-101 will automatically remove the used cap and put a new cap onto the magnetic probe before the final round of the capturing/washing/releasing cycle. Then an 8um filter was put over the final captured target cells and a capturing process performed, more than 95% of the unbound free microbeads were removed and the CTC were ready for observation under the microscope (FIG. 6, FIG. 10). These target cells can also be picked up individually for single cell downstream analyses. (FIG. 12, TAB. 1). The well position for capturing, washing, and releasing can be changed to different positions as desired.

### Example 4

### Target Cell Identification by Immunoassay and Gene Expression Patterns

A 3 mL of bone marrow sample was collected from a breast cancer patient in EDTA blood collection tube. The sample was filtered by 100 um membrane filter to remove the bone fragments and the rest of the sample then was incubated with 5 ul of EpCAM microbeads (Dynal) at room temperature for 1 hour with mild rotation. The bone marrow sample was then poured into the first well at one end of the first standard six-well plate. The other wells in the first standard six-well plate and the second standard six-well plate were filled with 10 mL PBS buffer solution. Set up QX Systems' CI-101 to perform super clean isolation Protocol. The CI-101 machine will automatically perform three rounds of capturing, washing, and releasing cycles and then the captured cells are released into the first release well. After the machine finishes two rounds of capturing/washing/releasing cycle, to avoid contamination by non-magnetic materials stuck onto the capture probe cap, CI-101 will automatically remove the used cap and put a new cap onto the magnetic probe before the final round of the capturing/washing/releasing cycle. One part of the collected tumor cells was incubated with DNase I (according to Life Tech manufacturer protocol) for 30 minutes and then the cells can either be used for immunostainin assay in the solution or alternatively be put on a glass slide, go through a process of air drying, fixation and then undergo the immunoassay for cell identification. CD45 antibody (labeled with TexasRed), CK antibodies (labeled with FITC), and DAPI (stains cell nuclear) were used in the immunostain method to identify tumor cells or blood cells (FIG. 11). Using the antibodies signatures, the cell type can be determined. For example, in this case, CD45 and DAPI positive cells will be considered as blood cells, on the other hand, CD45 negative, but DAPI and CKs positive cells will be considered as tumor cells (FIG. 11).

The remaining part of the collected tumor cells was used for the single tumor cell isolation test and subsequent molecular analyses. The collected cells were spread into the cell culture plate with PBS buffer solutions. A candidate for single EpCAM microbead bound tumor cell was picked up individually by pipette and put into a 100 uL PCR tube filled with limited PBS buffer solution. The individual cell was then analyzed using RNA transcription and cDNA application by CellDirect (Life Tech). The amplified cDNA was used for multiple genes detection analysis, including but are not limited to, detecting CD45 (blood cell specific gene), Cytokeratins, EpCAM, UBB (control gene). Using the gene expression signatures, the cell types can be determined. For example, in this particular case, the cells which expresses CD45 and UBB will be considered as blood cell, the cell which expresses UBB, CKs or/and EpCAM genes but not CD45 will be considered as a tumor cell (FIG. 12).

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

### REFERENCES

1. Ashworth, T. R (1869): A case of cancer in which cells similar to those in the tumors were seen in the blood after death. Australian Medical Journal 14: 146-7.
2. Cristofanilli, M. et al (2004): Circulating tumor cells, disease progression, and survival in metastatic breast cancer. NEJM 351:781-791.
3. Talasaz, A H et al (2009): Isolating highly enriched populations of circulating epithelial cells and other rare cells from blood using a magnetic sweeper device. PNAS USA 106: 3970-75.

TABLE 1

| Sample Name | UBB | CD45 | CKs | EpCAM |
|---|---|---|---|---|
| Ref-1 | 23.52 | 18.91 | 15.87 | 17.98 |
| Ref-2 | 23.47 | 18.36 | 15.56 | 19.09 |
| NTC-1 | 0 | 0 | 0 | 0 |
| NTC-2 | 0 | 0 | 0 | 0 |
| T47D-1 | 26.28 | 0 | 23.09 | 23.06 |
| T47D-2 | 25.95 | 0 | 22.54 | 22.32 |
| T47D-3 | 27.18 | 0 | 23.78 | 23.86 |
| SKBR3-1 | 27.01 | 0 | 0 | 24.65 |
| SKBR3-2 | 23.91 | 0 | 0 | 22.87 |
| SKBR3-3 | 23.46 | 0 | 0 | 21.87 |
| MDA231-1 | 25.35 | 0 | 18.17 | 17.67 |
| MDA231-2 | 22.61 | 0 | 12.66 | 20 |
| MDA231-3 | 25.24 | 0 | 17.17 | 18.48 |
| MCF7-1 | 24.61 | 0 | 20.13 | 22.05 |
| MCF7-2 | 24.63 | 0 | 21.12 | 22.9 |
| MCF7-3 | 21.65 | 0 | 17.63 | 18.06 |
| N-wbc-1 | 23.8 | 25.24 | 21.6 | 0 |
| N-wbc-2 | 25.12 | 26.42 | 22.73 | 0 |
| N-wbc-3 | 24.46 | 26.27 | 22.42 | 0 |
| CTC1 | 18.91 | 0 | 13.87 | 0 |
| CTC2 | 21.36 | 0 | 15.81 | 0 |
| CTC3 | 21.32 | 0 | 15.42 | 0 |
| DTC1 | 24.08 | 0 | 14.62 | 18.52 |
| DTC2 | 24.24 | 0 | 17.64 | 20.88 |
| DTC3 | 24.29 | 0 | 16.81 | 20.05 |

We claim:

1. A cell isolation method comprising:
(a) mixing target cells with a substance containing magnetic particles. (b) capturing the target cells onto a magnetic probe covered with a non-magnetic cap by turning on a capturing magnetic field; and
(c) releasing target cells from the cap by turning on a releasing magnetic field and turning off the capturing magnetic field.
(d) combination of the capturing, washing and releasing target cell steps for obtaining pure target cell needs.
(e) turning capturing magnetic field and releasing magnetic field ON or OFF for target cell capturing and releasing using various methods.

2. The cell isolation method as in claim 1, adding a washing step between (b) and (c) by putting the target cells into a buffer solution while the target cells are continuously held onto the cap during the washing step.

3. The cell isolation method as in claim 2, adding an additional step of repeating one or more of the capturing step, the washing step, or the releasing step, for one or more repeats.

4. The cell isolation method as in claim 1, where the magnetic probe comprises a magnetic rod which can move up to an up position and down to a down position inside the non-magnetic cap, generating the capturing magnetic field while at the down position; and the releasing magnetic comprises a magnetic field generated by a source underneath the cell culture plate with a magnetic polarity opposite to the polarity of the capturing magnetic rod.

5. The cell isolation method as in claim 4, where the capturing step comprises moving the probe in a sample well along a path which has a width that is slightly wider than the width of the probe.

6. The cell isolation method as in claim 5, where the washing step comprises moving the probe in a washing well along a path which has two or more distant sections for more effective washing.

7. The cell isolation method as in claim 1, where the target cell comprises a tumor cell, and substance containing magnetic microbeads comprises one or more types of antibody conjugated with microbeads.

8. The cell isolation method as in claim 7, where the antibodies conjugated with microbeads are cell specific antibodies. For example, EpCAM and MUC1 specifically bind to circulating tumor cells in the blood sample, CD45 specifically binds to blood cells, CD71 binds to fetal cells, CD44/24 bind to cancer stem cells.

9. A cell purification method, comprising steps as described in claim 1, adding a filtering step before or after any of the steps in claim 1 for one more times:
1) filtering out free unbound magnetic particles inside the substance containing magnetic particles by inserting a filter with the core size greater than the size of the free unbound magnetic particles and smaller than the size of the target cells such that the filter divides the well containing the target cells into two or more separate areas; and
2) separating the magnetic particles from the target cells by applying a magnetic field across the filter so that the magnetic particles pass through the cores of the filter and move out from the target cells.

10. The cell purification method as in claim 9, where the filter is put above the target sample; and the magnetic field across the filter is generated by switching on the capturing magnetic field and scanning the magnetic probe above the filter.

11. The cell purification method as in claim 9, where the filter is put below the target sample; and the magnetic field across the filter is generated by switching off the capturing magnetic field and switching on the releasing magnetic field.

12. The cell purification method as in claim 9, where the filter is rolled into a cylinder with the cylinder wall being the filter; and the magnetic field across the filter is generated by switching on the capturing magnetic field and scanning the magnetic probe outside the filter cylinder to attract any free unbound magnetic particles in the substance.

13. The cell purification method as in claim 9, filter pore size is bigger than free magnetic particles but smaller than target cells. For example, when the filter pore diameter is 8 um, the target cell has a diameter no less than 10 um, and the magnetic particles comprises microbeads measuring 4 um in diameter or less.

14. A cell labeling and identification method comprising:
(a) Collecting target cells;
(b) Performing immunostain assay on the captured cells for cell identification;
(c) Performing gene analysis to obtain gene signals comprising DNA or RNA patterns; and
(d) Determining whether the target cells are specific types of cells based on a combination of the presence or absence of one or more of the gene expressions.

15. The cell labeling and identification method as in claim 14, adding an addition step of analyzing single cell RNA using single cell lyses and cDNA synthesis and amplification.

16. The cell labeling and identification method as in claim 14, adding an additional step of analyzing the single cell using qRT-PCR methods.

17. A cell isolation device, comprising:
(a) a sample tank to hold samples containing target cells to be collected including magnetic particles; a wash tank for washing collected target cells; a releasing well for receiving collected target cells;
(b) a magnetic probe covered with a non-magnetic cap which can produce a capturing magnetic field at the tip of the cap; and
(c) a releasing magnetic source which produces a releasing magnetic field at the tip of the cap with an opposite polarity from the capturing magnetic field and capable of turning on and off the releasing magnetic field and the capturing magnetic field.

18. The cell isolation device as in claim 17, with a filter in one or more of the sample tank, wash tank, or release tank, where the filter divides the tank into two or more areas and the filter with the pores size smaller than the target cell and bigger than the magnetic particles.

19. The cell isolation device as in claim 17, all of the three tanks the width are wider than the width of the probe.

20. The cell isolation device as in claim 17, where collected cells are identified by its gene expression profiling, such as CD45, UBB, EpCAM, CKs and others.
